(19) United States
(12) Patent Application Publication
(10) Pub. No.: US 2008/0147020 A1
(43) Pub. Date: Jun. 19, 2008

(54) TEAR AWAY COLONIC SHEATH

(75) Inventor: Andrew M. Zwolinski, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/609,978

(22) Filed: Dec. 13, 2006

(51) Int. Cl. A61M 31/00

(52) U.S. Cl. 604/278; 600/101

(57) ABSTRACT

Methods and devices are provided for providing sterilize access to a body cavity through the wall of a body lumen. In one exemplary embodiment, a translumenal access device is provided having an outer elongate tubular member with an attachment housing located on a distal end thereof and adapted to form a seal with a tissue wall. The device can further include an inner sheath extending through a passageway of the outer elongate tubular member and adapted to isolate the sealed region of the tissue wall from the inner lumen in the elongate tubular member. The inner sheath can be removably mated to the device to allow any bacteria present within the sheath to be removed from the device after the sealed region of tissue is sterilized. The tubular member will be thus provide a sterile pathway which can be used to access a body cavity through the sealed tissue region.

### FIELD OF THE INVENTION

The present invention relates to methods and devices for performing surgical procedures, and in particular to a translumenal access device and to methods for accessing a body cavity through a body lumen.

### BACKGROUND OF THE INVENTION

Surgery through a natural orifice poses a number of challenges over laparoscopy or open surgery. Where the surgery is purely endoscopic, or strictly through a natural orifice, the surgeon faces the basic challenge of gaining adequate access to the desired operative site in the body. For example, many types of surgery require access to the peritoneum. The surgeon may reach the desired location, generally speaking, by penetrating the wall of a body lumen, such as a lumen of the gastrointestinal tract. For example, in a transanal procedure the surgeon typically creates a passageway through the wall of the colon to access the peritoneal cavity. Similarly, in a transoral or transgastric procedure the surgeon can create a passageway through the stomach.

Such procedures often involve creating a sterile access point or section of lumen (e.g., an otomy site) at which to penetrate the lumen wall. Because the otomy site and the peritoneal cavity, or other body cavity, can become inflamed and/or infected from microbial or other contamination, sterilization is often of utmost concern. Such concerns are heightened when accessing the peritoneum from the colon.

At the same time, in natural orifice surgery the surgeon faces a significant loss of dexterity and feel, and reduced ability to maneuver surgical tools and devices. Ideally, the access devices and techniques provide a stable and secure translumenal access point through which to operate, and do not unduly hinder the already difficult manipulation of surgical tools and devices into and within the body cavity.

Accordingly, there is a need in the art for novel methods and devices for providing access to a body cavity through the wall of a body lumen. There is also a need for devices and methods that can facilitate the sterilization of a local portion of a body lumen that can be used as an access point or otomy site for gaining access to a body cavity.

### SUMMARY OF THE INVENTION

The present invention generally provides methods and devices for providing sterile access to a body cavity via a body lumen. In one embodiment a translumenal access device is provided and includes an elongate tubular member having a passageway extending therethrough between a proximal end and a distal end thereof. The distal end can include an attachment housing adapted to form a seal with a tissue wall to isolate a sealed region of the tissue wall from surrounding tissue. The translumenal access device can further include an inner sheath extending through the elongate tubular member and having a distal end removably attached to the distal end of the elongate member, and an inner lumen extending therethrough and adapted to communicate with the sealed region of the tissue wall.

In one embodiment, the attachment housing can be adapted to apply suction to a tissue wall to form a seal with the tissue wall. For example, the attachment housing can have a substantially hollow housing having a plurality of suction ports formed therein. The device can also include a conduit having a proximal end located adjacent the proximal end of the elongate tubular member and adapted to couple to a vacuum source, and a distal end coupled to the attachment housing and adapted to deliver a suction force to the attachment housing to allow the attachment housing to form a seal with a tissue wall.

The present invention also provides various techniques for attaching the inner sheath to the elongate tubular member. In one embodiment, the distal end of the inner sheath is attached to the distal end of the elongate tubular member by an interference fit. In another embodiment, the distal end of the inner sheath is adhered to the distal end of the elongate tubular member and is adapted to tear away from the distal end of the elongate tubular member.

In yet another embodiment, the translumenal access device can include an actuation mechanism adapted to pull the distal end of the inner sheath away from the distal end of the elongate tubular member to detach the inner sheath from the elongate tubular member. For example, the actuation mechanism can be in the form of a loop coupled to the distal end of the inner sheath and movable between an initial position in which the loop is disposed around an inner surface of the elongate tubular member, and a retracted position in which the loop is substantially closed to substantially close the distal end of the inner sheath and thereby substantially seal the inner lumen extending through the inner sheath. The device can also include an elongate shaft having a proximal end located adjacent to a proximal end of the elongate tubular member, and a distal end attached to the loop. The elongate shaft can be adapted to move the loop between the initial and retracted positions. In another embodiment, the actuation mechanism can be in the form of a snare.

In another embodiment, a translumenal access device is provided having an outer elongate tubular member with a proximal end, a distal end, and a passageway extending therethrough between the proximal and distal ends. An attachment portion is located at the distal end of the outer elongate tubular member and it is adapted to attach to a tissue wall. The attachment portion can have an opening in fluid communication with the passageway of the outer elongate tubular member. The device can also include an inner elongate tubular member extending through the passageway of the outer elongate tubular member and having a proximal end positioned adjacent the proximal end of the outer elongate tubular member, a distal end positioned adjacent the attachment portion, and an inner lumen extending between the proximal and distal ends. The inner elongate tubular member can be adapted to form a barrier between the passageway extending through the outer elongate tubular member and fluid flowing through the inner lumen of the inner elongate tubular member and in fluid contact with a tissue wall attached to the attachment portion.

In one exemplary embodiment, the distal end of the inner elongate tubular member can be removably attached to at least one of the distal end of the outer elongate tubular member and the attachment portion. For example, the device can include a detachment mechanism coupled to an outer perimeter of the distal end of the inner elongate tubular member and adapted to detach the inner elongate tubular member and to substantially close the distal end of the inner elongate tubular member to substantially seal the inner lumen extending through the inner elongate tubular member. In another embodiment, the distal end of the inner elongate tubular member can be removably attached to at least one of the distal end of the outer elongate tubular member and the attachment portion by an adhesive.

Exemplary methods for providing sterile access to a body cavity via a body lumen are also provided, and in one embodiment the method can include inserting an elongate tubular member through a body lumen to position a distal end of the elongate tubular member adjacent to a tissue wall to be penetrated through to gain access to a body cavity. A seal can be formed between the distal end of the elongate tubular member and the tissue wall to isolate a sealed region of the tissue wall that is in communication with an inner lumen of an inner sheath extending through the elongate tubular member and having a distal end coupled to the distal end of the elongate tubular member. The seal can be formed, for example, by a applying suction force to an attachment housing coupled to the distal end of the elongate tubular member. The sealed region of the tissue wall can be sterilized, and the inner sheath can be removed from the elongate tubular member such that the elongate tubular member provides a sterile passageway to the sealed region of the tissue wall. In an exemplary embodiment, the sealed region of the tissue wall is sterilized by flushing a fluid through the inner sheath to flush bacteria from the sealed region of the tissue wall. The inner sheath can form a barrier between a passageway extending through the elongate tubular member and the fluid.

In another embodiment, the method can include removing the inner sheath by constricting the distal end of the inner sheath to substantially close the distal end of the inner sheath and effectively seal the inner lumen extending through the inner sheath. This can be achieved, for example, by pulling on a loop coupled to the distal end of the inner sheath to substantially close the loop. In other aspects, the method can include piercing the sealed region of the tissue wall to provide access through the sterile passageway of the elongate tubular member to a body cavity. Once access is obtained, various instruments, such as an endoscope, can be inserted through the sterile passageway and into the body cavity.

### BRIEF DESCRIPTION OF THE DRAWINGS

The invention will be more fully understood from the following detailed description taken in conjunction with the accompanying drawings, in which:

FIG. 1 is a side view of one embodiment of a distal portion of a translumenal access device having an elongate tubular member, an attachment housing and an inner sheath;

FIG. 2 is a perspective view of the attachment housing of FIG. 1, showing a conduit coupled thereto and extending along the elongate tubular member;

FIG. 3A is a perspective view of the inner sheath of FIG. 1, showing an actuation mechanism coupled to the inner sheath for moving the inner sheath between an initial position and a contracted position; and

FIG. 3B is a cross-sectional view of the device of FIG. 1, showing the inner sheath in the contracted position with a distal end of the inner sheath being constricted and substantially closed by the actuation mechanism;

FIG. 4 is a partially cross-sectional view of the device of FIG. 1, showing the attachment housing attached to the tissue wall and an endoscope extending through the device and the tissue wall.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for providing sterile access to a body cavity via a body lumen. For example, the methods and devices can be used to create a sterile passageway or access point through the colon and into the peritoneum. The sterile passageway is particularly advantageous as it provides a path that can be repeatedly used to introduce an instrument, such as an endoscope, through a body lumen and into a body cavity without carrying bacterial flora of the body lumen into the body cavity, thereby reducing the risk of infections.

A person skilled in the art will appreciate that, while the methods and devices are described in connection with endoscopic procedures, the methods and devices disclosed herein can be used in numerous surgical procedures. By way of non-limiting example, the devices can be used in laparoscopic procedures, in which the device is introduced percutaneously. The methods and devices can also be used in open surgical procedures.

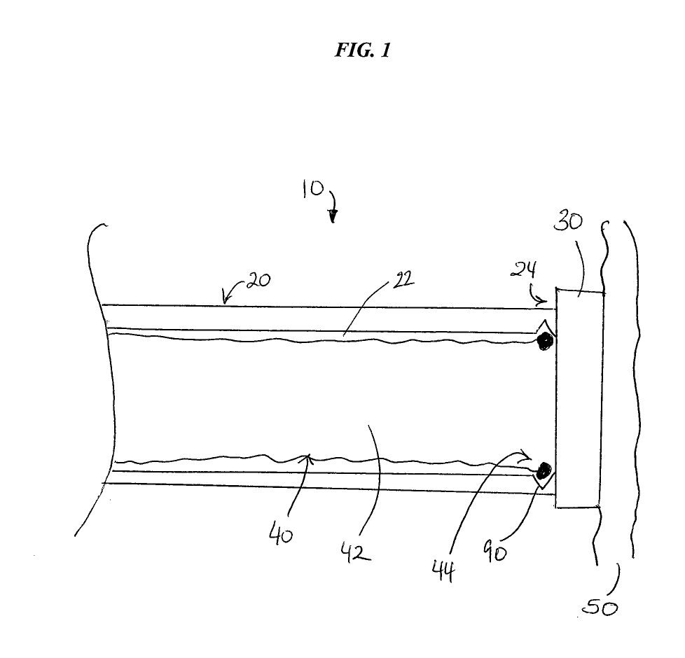

FIG. 1 illustrates one exemplary embodiment of a translumenal access device 10. As shown, the device 10 generally includes an elongate tubular member 20 having a proximal end (not shown), a distal end 24, and a passageway 22 extending longitudinal therethrough between the proximal and distal ends. FIG. 1 also illustrates an attachment housing 30 that is coupled to the distal end 24 of the elongate tubular member 20, and that is adapted to form a seal with a tissue wall 50. The device 10 also includes an inner sheath 40 extending through the passageway 22 of the elongate tubular member 20, and having a proximal end (not shown), a distal end 44, and an inner lumen 42 extending therethrough. The inner lumen 42 of the sheath 40 can be configured to communicate with the sealed region of tissue wall 50. In use, the attachment housing 30 can form a seal with a tissue wall 50. The sealed region of tissue can then be sterilized, e.g. by flushing fluid through the inner lumen 42 of the sheath 40. Once the tissue is sterilized, the inner sheath 40 can be removed to remove any remaining bacteria, thereby providing a sterile pathway through the elongate tubular member 20 to the tissue. One or more tools can thus be introduced through the tubular member 20 to puncture through the sealed region of tissue and to perform various procedures in the body cavity accessed via the puncture. The device 10 can also include a variety of other features to facilitate use of the device 10 such as an actuation mechanism for removing the inner sheath 40.

The elongate tubular member 20 can have a variety of configurations, but is preferably configured such that the proximal end (not shown) can remain outside a patient's body. While the distal end 24 is positioned within the patient's body, preferably through a body lumen. The particular configuration of the elongate tubular member 20 can vary depending on the type of procedure being formed. For example, the elongate tubular member 20 can be rigid or flexible, and it can vary in shape, size and length. In an exemplary embodiment, at least a portion of the elongate tubular member 20 is flexible to allow the elongate tubular member 20 to be inserted endoscopically through a natural orifice and through a tortuous body lumen. In one embodiment, the elongate tubular member 20 can be at least partially formed of a flexible, malleable or pliable material to allow for reshaping or bending. Exemplary materials include, by way of non-limiting example, metals such as aluminum, stainless steel or titanium; shape-memory materials; plastics; or various other biocompatible materials.

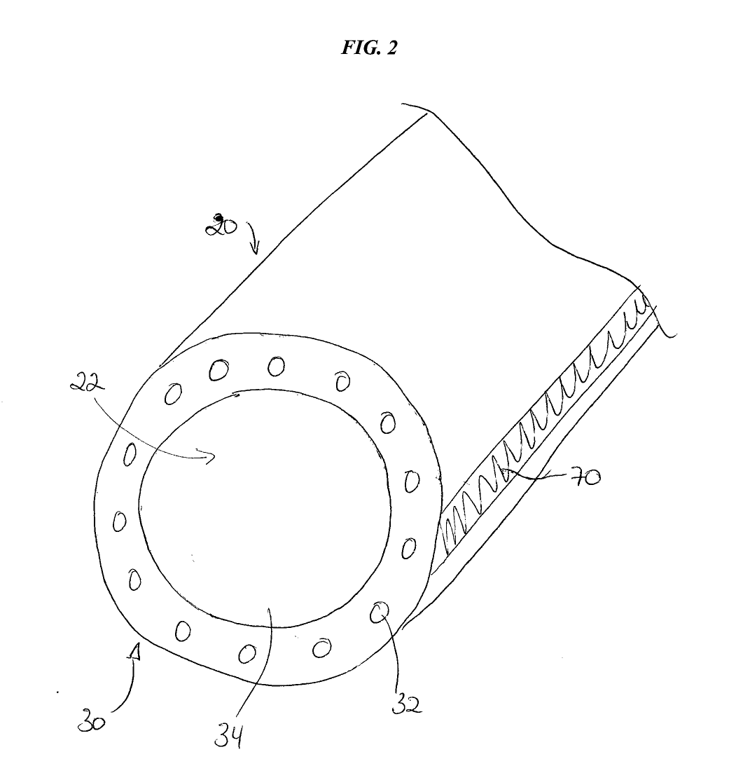

FIG. 2 illustrates a more detailed view of the attachment housing 30 of the device 10. In the illustrated embodiment, the attachment housing 30 has a substantially hollow cylindrical shape with an opening 34 formed therethrough and in communication with the passageway 22 of the elongate tubular member 20. The attachment housing 30 can, however, have a number of other shapes and configurations, including an elliptical, square, or rectangular shape, or any other shape or configuration. FIG. 2 further illustrates the attachment housing 30 mated to the distal-most end of the elongate tubular member 20. The attachment housing 30 can be mated to the elongate tubular member 20 in a variety of ways, for example, the attachment housing 30 can be integrally formed with the distal end 24 of the elongate tubular member 20, or it can be removably attached thereto.

The attachment housing 30 can also be adapted to removably mate to and form a seal with a tissue wall (not shown). A number of techniques can be used to facilitate such mating with a tissue wall. In an exemplary embodiment, the attachment housing 30 can be adapted to mate with the tissue wall by suction. As shown in FIG. 2, the attachment housing 30 can include a plurality of ports 32 formed in a distal-facing surface thereof. The ports 32 allow for a suction force to be applied therethrough, enabling the attachment housing 30 to engage a tissue wall. The distal end of the attachment housing 30 can, however, have a variety of other configurations that allow the attachment housing 30 to apply a suction force to a tissue wall. For example, the distal end of the attachment housing 30 include a mesh material that allows a suction force to be applied therethrough.

Various techniques can also be used for delivering a suction force to the attachment housing 30. In an exemplary embodiment, the device 10 can include a conduit 70 extending longitudinally along the elongate tubular member 20. It should be understood that the conduit 70 can extend along an external or internal surface, or it can be embedded in the sidewalls of the elongate tubular member 20. The conduit 70 can also be in the form of a lumen extending through the sidewall of the device 10. The proximal end (not shown) of the conduit 70 is preferably adapted to mate to a source that provides a suction force, such as, for example, a vacuum source. The distal end of the conduit 70 can be in communication with the hollow interior of the attachment housing 30. In use, the conduit 70 can deliver suction to the ports 32 thereby enabling the attachment housing 30 to engage with a tissue wall and form a seal therewith.

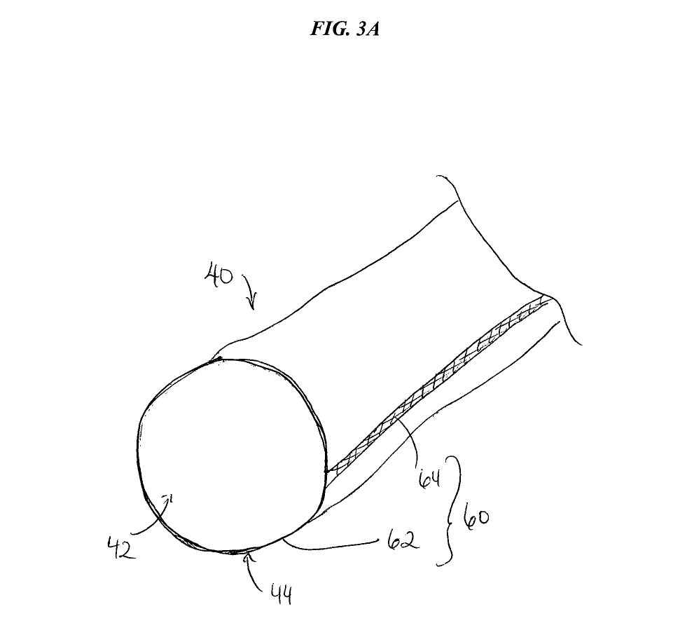

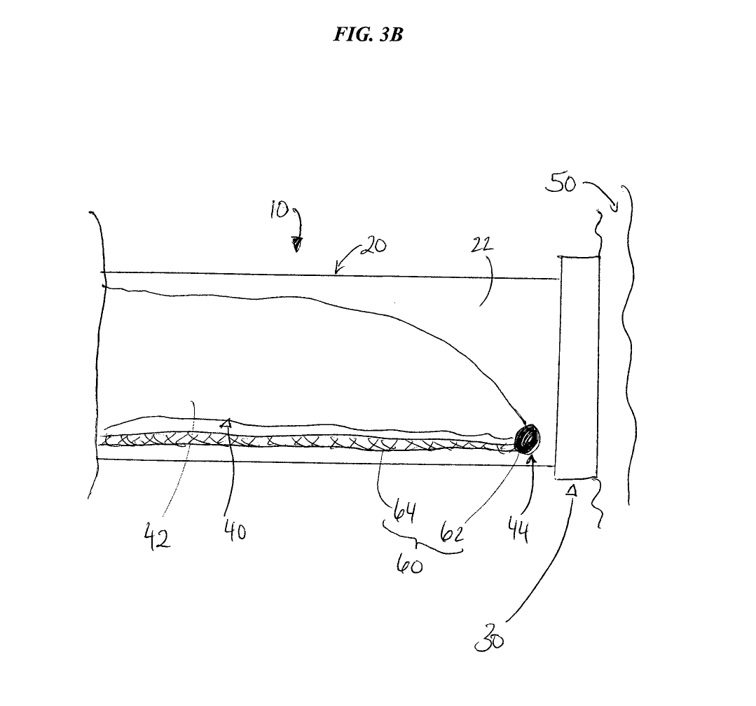

As previously indicated, the device 10 can also include an inner sheath 40 extending through the passageway 22 of the elongate tubular member 20. While the inner sheath 40 can have a variety of configurations, in an exemplary embodiment the inner sheath 40 is configured to form a barrier between a tissue wall sealed within the attachment housing 30 and the inner wall of the elongate tubular member 20. In an exemplary embodiment, as shown in FIG. 3A, the inner sheath 40 can have a distal end 44 that is coupled to the attachment housing 30 and a proximal end (not shown) that extends out of the proximal end of the elongate tubular member 20. An inner lumen 42 can extend through the inner sheath 40 for receiving tools and/or materials, such as a sterilization fluid. The shape of the inner sheath 40 can also vary, but preferably has a shape that corresponds with the shape of the passageway 22 of the elongate tubular member 20 when the inner sheath 40 is disposed therein. The inner sheath 40 can also be formed of a variety of materials, including flexible, malleable, or pliable materials that allow for reshaping or bending.

As indicated above, the distal end 44 of the inner sheath 40 can be removably mated to the attachment housing 30 or to the distal end 24 of the elongate tubular member 20. In one embodiment, the removable engagement between the distal end 44 of the sheath 40 and the distal end 24 of the elongate tubular member 20 can be achieved by using an interference fit. For example, the distal end 44 of the sheath 40 can include an annular lip or rim formed thereon that is adapted to fit within a groove 90 formed in an inner surface of the elongate tubular member 20 to permit the distal end 44 to be removably engaged by the groove 90, as shown in FIG. 1. While not shown, the groove 90 can alternatively be formed around an inner sheath of the attachment housing 30.

The detachment and removal of the inner sheath can be achieved in various ways. In one embodiment, a force can be applied to the proximal end of the sheath 40 to merely pull the rim out of the groove 90 thereby removing the sheath 40 from the elongate tubular member 20. In an exemplary embodiment, however, the device 10 preferably includes an actuation mechanism 60 that is adapted to close and substantially seal the distal end 44 of the sheath 40 while detaching the sheath 40 from the device 10. FIG. 3A illustrates one exemplary embodiment of an actuation mechanism 60 coupled to an outer perimeter of the distal end 44 of the inner sheath 40. As shown, the actuation mechanism 60 includes a loop 62 disposed around the distal end 44, and an elongate shaft or cable 64 attached to the loop 62 and extending therefrom. The cable 64 can extend from the distal end 44 to the proximal end (not shown) of the device 10. In use, as illustrated in FIG. 3B, a pulling force can be exerted on the cable 64 to cause the loop 62 to move from an initial, open position to a second, closed or retracted position, as shown, such that the distal end 44 of the inner sheath 40 is constricted and substantially closed. A person skilled in the art will appreciate that the loop 62 can form the lip or rim of the sheath 40, thus allowing the distal end 44 of the sheath 40 to be held in the groove 90 by an interference or compression fit. The loop 62 is, however, preferably movably coupled to the sheath 40 to allow the loop 62 to slide and close the distal end 44 of the sheath 40.

In another exemplary embodiment, the loop 62 can be in the form of an elastic band or a ring formed of a shape memory material. In use, the elastic band can be held in the open position by the groove 90, and the cable 64 can be pulled to remove the elastic band or ring from the groove 90, thereby allowing the ring or elastic band to self-close and thereby constrict and substantially close the distal end 44 of the inner sheath 40.

Other techniques can be used for detaching and removing the inner sheath 40 from the device 10. For example, the distal end 44 of the inner sheath 40 can be adhered to the distal end 24 of the elongate tubular member 20 with an adhesive that allows the sheath 40 to be torn away from the device 10. The adhesive can also be a degradable adhesive. In use, when the adhesive degrades, the distal end 44 of the inner sheath 40 collapses upon itself, and thus the sheath 40 will substantially close. In another embodiment, the distal end 44 of the inner sheath 40 can be substantially closed by creating a vacuum force within the inner lumen 42 of the sheath 40 that is sufficient to cause the distal end 44 of the inner sheath 40 to collapse to a closed position. In a yet another embodiment, an instrument, such as an endoscope, can be inserted through the inner sheath 40 and a pair of graspers can be deployed through the endoscope to grasp an inner surface of the distal end 44 of the inner sheath 40 and pull the distal end 44 closed. The graspers or other removal tools can also be directly passed through the sheath 40 without using an endscope.

The present invention also provides methods for providing sterile access to a body cavity via a body lumen. In one exemplary embodiment, a translumenal access device, such as device 10 described above, can be inserted through a body lumen such as the esophagus or colon. The distal-most surface of the attachment housing 30 can be positioned adjacent a tissue wall 50, as shown in FIG. 1. A vacuum force can be applied to the conduit 70 to create a vacuum force within the attachment housing 30. The application of the vacuum force will cause the ports 32 in the attachment housing 30 to suction the tissue wall 50 onto the housing 30, thereby forming a seal with the tissue wall 50 to isolate a region of the tissue located within the sealed region.

Once the device 10 is attached to the tissue wall 50, the sealed region of tissue wall can be sterilized using various techniques know in the art. For example, a fluid, such as saline, can be flushed through the inner lumen 42 of the inner sheath 40 to cleanse the isolated tissue region. Following sterilization of the sealed tissue region, the inner sheath 40 can be removed. For example, the actuation mechanism 60 can be activated to move the distal end 44 of the sheath 40 from an initial, open position to a constricted position, thereby substantially closing the inner sheath 40, as shown in FIG. 3B. This will ensure that any bacterial matter remaining in the inner sheath 40 will be removed from the device 10, leaving a sterile passageway 22 through the outer elongate tubular member 20. Once the distal end 44 of the inner sheath 40 is sealed, a pulling force is exerted upon the actuation mechanism 60 or the inner sheath 40 to remove the sheath 40 from elongate tubular member 20.

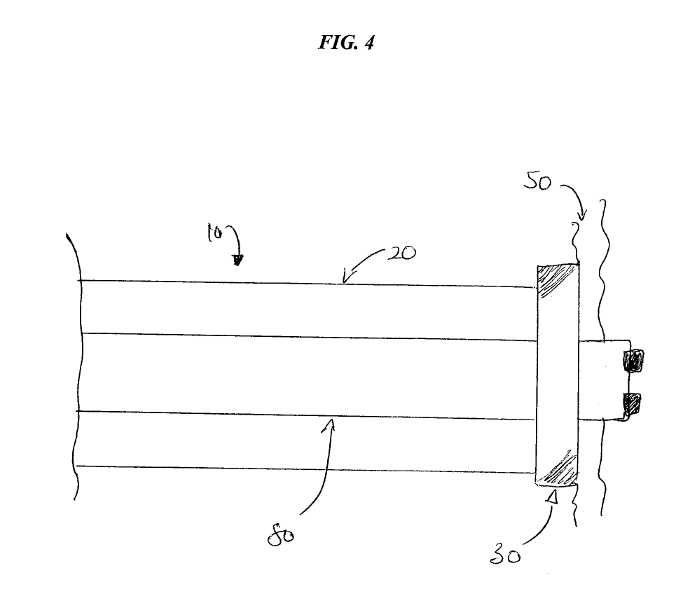

As illustrated in FIG. 4, once the inner sheath 40 has been from removed, a medical instrument, such as an endoscope 80, can then be inserted through the sterile passageway 22 of the outer elongate tubular member 20 to position the distal end of the endoscope 80 adjacent to or within the opening 34 of the attachment housing 30. A tool can be inserted through the endoscope 80 to form a puncture or cut in the tissue wall 50, thereby providing access to an adjacent body cavity. The method of the present invention thus provides a sterilize passageway and access point, which can be repeatedly used and which prevents flora of a body lumen from entering the adjacent body cavity thus reducing the likelihood of infection.

The device disclosed herein can also be designed to be disposed of after a single use, or it can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A translumenal access device, comprising:
an elongate tubular member having a passageway extending therethrough between a proximal end and a distal end, the distal end having an attachment housing adapted to form a seal with a tissue wall to isolate a sealed region of the tissue wall from surrounding tissue; and
an inner sheath extending through the elongate tubular member and having a distal end removably attached to the distal end of the elongate member, and an inner lumen extending therethrough and adapted to communicate with the sealed region of the tissue wall.

2. The device of claim 1, wherein the attachment housing is adapted to apply suction to a tissue wall to form a seal with the tissue wall.

3. The device of claim 2, wherein the attachment housing comprises a substantially hollow housing having a plurality of suction ports formed therein.

4. The device of claim 3, further comprising a conduit having a proximal end located adjacent the proximal end of the elongate tubular member and adapted to couple to a vacuum source, and a distal end coupled to the attachment housing and adapted to deliver a suction force to the attachment housing to allow the attachment housing to form a seal with a tissue wall.

5. The device of claim 1, wherein the distal end of the inner sheath is attached to the distal end of the elongate tubular member by an interference fit.

6. The device of claim 5, further comprising an actuation mechanism adapted to pull the distal end of the inner sheath away from the distal end of the elongate tubular member to detach the inner sheath from the elongate tubular member.

7. The device of claim 1, wherein the distal end of the inner sheath is adhered to the distal end of the elongate tubular member and is adapted to tear away from the distal end of the elongate tubular member.

8. The device of claim 7, further comprising an actuation mechanism adapted to pull the distal end of the inner sheath away from the distal end of the elongate tubular member to detach the inner sheath from the elongate tubular member.

9. The device of claim 8, wherein the actuation mechanism comprises a loop coupled to the distal end of the inner sheath and movable between an initial position in which the loop is disposed around an inner surface of the elongate tubular member, and a retracted position in which the loop is substantially closed to substantially close the distal end of the inner sheath and thereby substantially seal the inner lumen extending through the inner sheath.

10. The device of claim 9, further comprising an elongate shaft having a proximal end located adjacent to the proximal end of the elongate tubular member, and a distal end attached to the loop, the elongate shaft being adapted to move the loop between the initial and retracted positions.

11. The device of claim 8, wherein the actuation mechanism comprises a snare.

12. A translumenal access device, comprising:
an outer elongate tubular member having a proximal end, a distal end, and a passageway extending therethrough between the proximal and distal ends;
an attachment portion located at the distal end of the outer elongate tubular member and adapted to attach to a tissue wall, the attachment portion having an opening in fluid communication with the passageway of the outer elongate tubular member; and
an inner elongate tubular member extending through the passageway of the outer elongate tubular member and having a proximal end positioned adjacent the proximal end of the outer elongate tubular member, a distal end positioned adjacent the attachment portion, and an inner lumen extending between the proximal and distal ends, the inner elongate tubular member adapted to form a barrier between the passageway extending through the outer elongate tubular member and fluid flowing through the inner lumen of the inner elongate tubular member and in fluid contact with a tissue wall attached to the attachment portion.

13. The device of claim 12, wherein the distal end of the inner elongate tubular member is removably attached to at least one of the distal end of the outer elongate tubular member and the attachment portion.

14. The device of claim 12, further comprising a detachment mechanism coupled to an outer perimeter of the distal end of the inner elongate tubular member and adapted to detach the inner elongate tubular member and to substantially close the distal end of the inner elongate tubular member to substantially seal the inner lumen extending through the inner elongate tubular member.

15. The device of claim 12, further comprising a conduit having a proximal end located adjacent the proximal end of the outer elongate tubular member and adapted to couple to a vacuum source, and a distal end coupled to the attachment portion and adapted to deliver a suction force to the attachment portion to allow the attachment portion to form a seal with a tissue wall.

16. The device of claim 12, where the distal end of the elongate tubular member is removably attached to at least one of the distal end of the outer elongate tubular member and the attachment portion by an adhesive.

17. A method for providing sterile access to a body cavity via a body lumen, comprising:
inserting an elongate tubular member through a body lumen to position a distal end of the elongate tubular member adjacent to a tissue wall to be penetrated through to gain access to a body cavity;
forming a seal between the distal end of the elongate tubular member and the tissue wall to isolate a sealed region of the tissue wall, the sealed region of the tissue wall being in communication with an inner lumen of an inner sheath extending through the elongate tubular member and having a distal end coupled to the distal end of the elongate tubular member;
sterilizing the sealed region of the tissue wall; and
removing the inner sheath from the elongate tubular member such that the elongate tubular member provides a sterile passageway to the sealed region of the tissue wall.

18. The method of claim 17, wherein sterilizing the sealed region of the tissue wall comprises flushing a fluid through the inner sheath to flush bacteria from the sealed region of the tissue wall, the inner sheath forming a barrier between a passageway extending through the elongate tubular member and the fluid.

19. The method of claim 17, wherein removing of the inner sheath comprises constricting the distal end of the inner sheath, to substantially close the distal end of the inner sheath and effectively seal the inner lumen extending through the inner sheath.

20. The method of claim 19, wherein constricting of the distal end of the inner sheath comprises pulling on a loop coupled to the distal end of the inner sheath to substantially close the loop.

21. The method of claim 17, wherein forming a seal between the distal end of the elongate tubular member and the tissue wall is achieved by a applying suction force to an attachment housing coupled to the distal end of the elongate tubular member.

22. The method of claim 17, further comprising piercing the sealed region of the tissue wall to provide access through the sterile passageway of the elongate tubular member to a body cavity.

23. The method of claim 22, further comprising inserting an endoscope through the sterile passageway and into the body cavity.
